(19) United States
(12) Patent Application Publication
(10) Pub. No.: US 2014/0180596 A1
(43) Pub. Date: Jun. 26, 2014

(54) EFFECTIVE ROOT ZONE USE IN CROP MANAGEMENT

(71) Applicant: PURESENSE ENVIRONMENTAL INC., Oakland, CA (US)

(72) Inventor: Michelle M. Frey, Oakland, CA (US)

(73) Assignee: PURESENSE ENVIRONMENTAL INC., Oakland, CA (US)

(21) Appl. No.: 14/045,593

(22) Filed: Oct. 3, 2013

Related U.S. Application Data
(63) Continuation of application No. PCT/US12/32611, filed on Apr. 6, 2012.
(60) Provisional application No. 61/473,002, filed on Apr. 7, 2011.

(51) Int. Cl. G01N 33/00

(52) U.S. Cl. CPC G01N 33/0098
USPC 702/19

(57) ABSTRACT

Embodiments of the present disclosure relate generally to nutrient monitoring in an agricultural field, and more specifically to systems, devices, and methods for tracking the fate and transport of nitrogen in agricultural environments and for determining an effective root zone of a crop.

### RELATED APPLICATIONS

This application is a continuation of PCT Appl. No. PCT/US2012/032611, filed Apr. 6, 2012, which claims priority to U.S. Provisional Application Ser. No. 61/473,002 filed Apr. 7, 2011, which is incorporated herein by reference in its entirety.

### TECHNICAL FIELD

The present disclosure relates generally to systems, devices, and methods for using an effective root zone in crop management.

### BACKGROUND

Managing nitrogen fate and transport in agricultural settings is critical to the success and sustainability of farmers today and into the future. Farmers are challenged by increased pressure to mitigate nitrogen contamination of surface waters and underlying groundwaters while also challenged to meet marketplace pressure for remaining competitive in their crop production practices. Nitrogen represents the key to these challenges as the environmental consequences of its contamination of surface and ground waters are severe while also being the main nutrient required for crop production.

Further, the fate of nitrogen in agricultural settings is complex due to the variety of chemical forms and states that nitrogen can take in such settings. When applied to the field, nitrogen is typically in a liquid form as a mixture of organic and inorganic compounds. Organic forms of nitrogen, typically present as urea (CH₄N₂0), are used when a farmer wants to have nitrogen remain resident in the soil profile beyond the time period of the initial fertilizer application. Organic forms of nitrogen will sorb onto the surface of soil particles when introduced by the irrigation water infiltrating into the soil profile. Over time and with subsequent irrigation events, these compounds are oxidized and form the more soluble inorganic forms of nitrogen, namely nitrate (N0₃−), nitrite (N0₂−), and ammonia (NH₄+).

Plants uptake nitrogen that is in an inorganic form through the plant's root system, specifically as nitrate and nitrite. Thus, the sorption of organic nitrogen to soils enables on-going release of nitrogen for plant uptake over time. The concerns about environmental contamination from nitrogen into ground waters are focused on the release and migration of inorganic nitrogen as it is far more mobile in the soil environment and can be transported to aquifers due to excess irrigation water applied to the fields. So, while the transformation of nitrogen sorbed onto soil particles is essential for the on-going delivery of nutrients to the crop, it is also the potential source of nitrogen responsible for environmental contamination.

Nutrient management programs in agriculture can benefit from decision support and control systems relating to crop nitrogen uptake over time versus the total nitrogen required for crop production, nitrogen release beyond the crop root zone over time that could contribute to environmental contamination concerns, and automated operation of irrigation pump stations, including the addition of fertilizer compounds, based on the determination of additional nitrogen requirements at given points in time.

Improved systems, devices, and methods are needed to satisfy these and other related goals in the industry.

### SUMMARY

Example embodiments of systems, devices, and methods for determining an effective root zone for a crop and using that effective root zone in crop management are provided herein. Determination of the effective root zone allows a grower to, for example, assess the degree at which plant uptake of nitrogen has occurred. This, in turn, can allow one to track nitrogen fate after its application to a field for the purposes of optimizing application of nitrogen-based fertilizers for crop production and/or minimizing the potential environmental degradation from “off-farm” migration of harmful nitrogen compounds.

Example embodiments of systems, devices, and methods for determining the nitrogen inputs and transport effects leading to the conversion and subsequent availability of nitrogen in forms suitable for crop use are also provided herein. This includes systems, methods, and devices where data is captured from field measurement devices, delivered to centralized computer services, processed and analyzed, and provided to end users in forms that are understandable for making subsequent nutrient application decisions.

Other systems, devices, methods, features and advantages of the subject matter described herein will be or will become apparent to one with skill in the art upon examination of the following figures and detailed description. It is intended that all such additional systems, devices, methods, features and advantages be included within this description, be within the scope of the subject matter described herein, and be protected by the accompanying claims. In no way should the features of the example embodiments be construed as limiting the appended claims, absent express recitation of those features in the claims.

### BRIEF DESCRIPTION OF THE DRAWINGS

The accompanying drawings, which are incorporated into this specification, illustrate one or more example embodiments and, together with the detailed description, serve to explain principles and example implementations. One of skill in the art will understand that the drawings are illustrative only, and not necessarily to scale, and that what is depicted therein may be adapted based on the text of the specification and the spirit and scope of the teachings herein.

FIG. 1 is a schematic representation of “just in time” decision support for nitrogen management.

FIG. 2 is schematic representation of an agricultural field and soil environment.

FIG. 3 is a flowchart showing nitrogen balance methods for certain management measures.

FIG. 4 is a representation of an example embodiment of an agricultural monitoring system.

FIG. 5 is a side, cut away view of an example embodiment of a nutrient monitoring device.

FIG. 6 is a schematic view of an example embodiment of a control panel.

FIG. 7 is a flowchart illustrating one example of a method for determining the effective root zone (ERZ) in a particular soil profile.

FIG. 8 is a flow chart illustrating an example high level nutrient monitoring method.

### DETAILED DESCRIPTION

Various example embodiments of systems, devices, and methods are described in the context of determining an effective root zone for a crop and using the effective root zone determination in the management of water and nutrient application to that crop. The present disclosure also focuses on the fate and transport of nitrogen in a soil profile, but this disclosure is not limited to nitrogen, and one of ordinary skill in the are will readily recognize that the disclosure is equally applicable to the fate and transport of other nutrients or even water.

Those of ordinary skill in the art will understand that the following detailed description is illustrative only and is not intended to be in any way limiting. Other embodiments may suggest themselves to such skilled persons having the benefit of this disclosure and the teachings provided herein.

In the interest of clarity, not all of the routine features of the described example embodiments are shown or discussed. It will of course be appreciated that in the implementation of any of these embodiments, decisions must be made in order to achieve the specific goals of the implementer, such as compliance with cost, business-related, regulatory, safety, social, environmental, health, and other constraints, and that these specific goals will vary from one implementation to another.

Growers need to manage their crop's nutrition requirements throughout the production cycle. Over the time that a crop grows, a grower typically has only one or two measurement events through the production cycle to make decisions about nutrient applications. This means that the decision process, in conventional situations, is poorly supported during critical period of crop growth and development.

Grower decisions about nutrition needs are derived from a conservative practice that assures plentiful nutrients are available for plant uptake. However, growers have no concrete methods by which to determine whether their practices are adequate or can lead to harm of either their crop or the environment.

Nitrogen, for example, is one of the most important macronutrients in the production of crops. However, the consequences of over-applying nitrogen-based fertilizers to agricultural fields, though, can include contamination of underlying aquifer supplies with excessive levels of nitrate and nitrite as well as the release of nitrous oxide (NOX) gases to the atmosphere.

The present subject matter provides growers with (preferably real-time) analytical tools that will track nitrogen fate in the agricultural environment so that more accurate and informed decisions regarding fertilizer use can be made and so that more environmentally appropriate practices to manage fertilizer applications and fate may be implemented. These tools make nutrition management more “just-in-time” by tracking the sources of nitrogen, and the fate and transport of that nitrogen in the field environment. For example, through a time series analysis of data regarding nitrogen fate and transport, growers can be provided with information regarding their crop's nutrition requirements and the sufficiency of the available nitrogen in meeting the crop's demand.

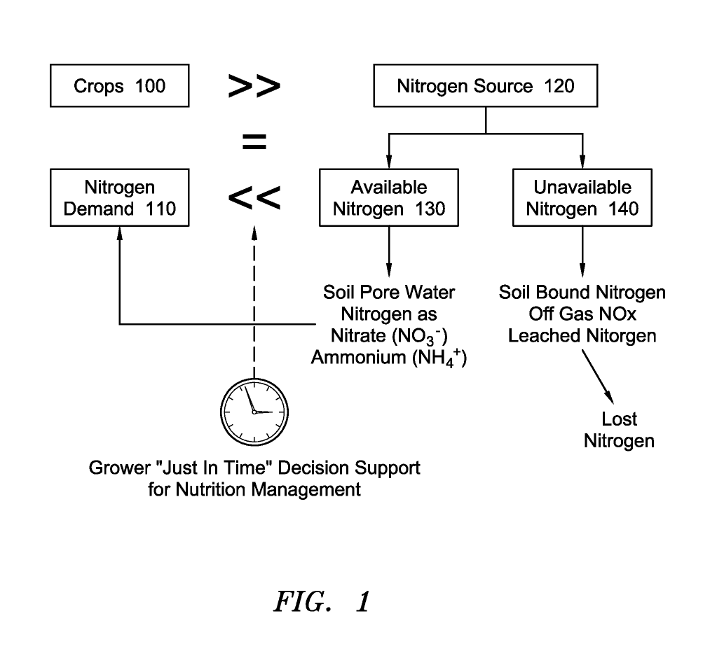

FIG. 1 is a simplified schematic representation of the “just-in-time” concept of the invention. As illustrated, crops 100 have a certain nitrogen demand 110. Nitrogen sources 120 are tracked and are comprised of available nitrogen 130 and unavailable nitrogen 140. Available nitrogen 130 is derived from a variety of means, such as but not limited to nitrogen components present in soil pore water. Nitrogen components in the soil pore water are typically present as nitrate (NO₃) and ammonium (NH₄). Unavailable nitrogen 140 is generally comprised of soil bound nitrogen, off gas NOx, and leached nitrogen. Using embodiments described herein, one can determine the available nitrogen present in a certain soil profile or environment. Growers can then determine if the available nitrogen 130 is sufficient to meet the nitrogen demand 110 of their crops 100.

In some embodiments, capture of real-time monitoring data through the soil profile of agricultural fields is achieved. Examples of systems for real-time monitoring soil moisture in-situ in agricultural fields that may be used with the present embodiments are described in detail in International Application No. PCT/US12/27588, filed Mar. 2, 2012, and entitled “Systems, Devices, and Methods for Environmental Monitoring in Agriculture,” the disclosure of which is hereby incorporated by reference in its entirety.

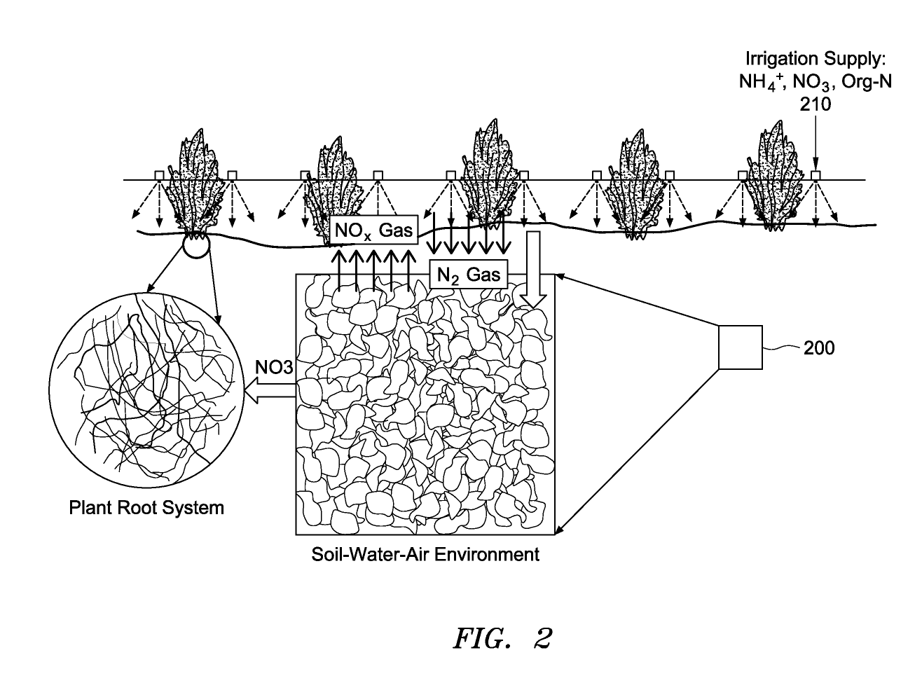

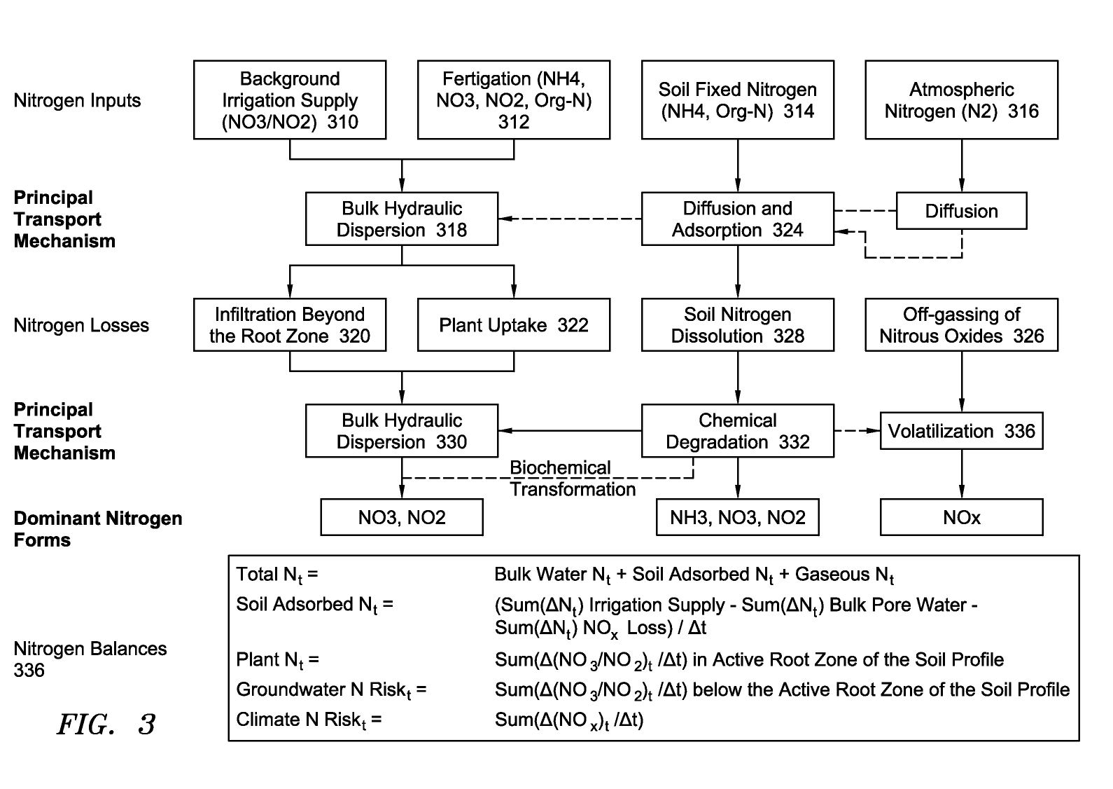

In an agricultural setting, a “nitrogen system” may be defined as having nitrogen inputs and nitrogen losses within a given soil environment 200. The fate and transport, and/or flux, of nitrogen compounds in the defined soil environment 200 is analyzed. FIG. 2 is a schematic representation of a nitrogen system in an agricultural setting, and FIG. 3 is a flowchart illustrating nitrogen inputs, nitrogen flux, and nitrogen losses within a nitrogen system. As shown in FIGS. 2-3, the major source of nitrogen input is through the irrigation supply 210 either as background nitrogen levels 310 or through common fertigation 312 practices for the addition of nutrients for crop production (sometimes collectively referred to as “applied nitrogen”). The background irrigation supply 310 generally includes NO₃/NO₂. Fertigation 312 generally includes the application of any one of more of: NH₄, NO₃, NO₂ or organic-N. Minor sources of nitrogen input may include soil fixed nitrogen 314 (comprised of NH₄ and organic-N) and atmospheric nitrogen 316 (N₂). The various nitrogen inputs are graphically shown in FIG. 2.

Soil environments 200 shown in FIG. 2, include a soil-water-air environment, which can be referred to as a “soil profile.” Using bulk water or hydraulic dispersion 318, the applied nitrogen migrates through soil pore water either laterally or by drainage through the vertical profile of the soil. Soil pore water present within the soil environment 200 is broadly defined as water that is found to occupy the void spaces between and around soil particles. Once in the soil profile, the nitrogen may remain in the soil pore water and continue to migrate away from the effective root zone of the crop 320, the nitrogen may be uptaken by the roots of the crop and thereby transformed by the crop 322, the nitrogen may adsorp to the soil surface (only appreciably true for Org-N and NH4+forms of nitrogen) 324, or the nitrogen may be transformed into a gaseous form of nitrogen and either lost to the atmosphere 326 or retained within the soil pore volume 328 (often as NOx).

As long as the nitrogen remains in the soil-water-air environment, the various forms of nitrogen will be continuously transformed interchangeably to maintain equilibrium conditions. Diffusion and biochemical reactions 332 begin to dominate the main fate and transport mechanisms.

During the primary crop production period when fertilizer applications are routinely performed, the maximum holding period for equilibrium among nitrogen forms to be achieved are very limited (hours to days). Therefore, the main paths for fate and transport mechanisms remain the bulk water dispersion 330. However, over time, substantial contributions of nitrogen volatilization 334 can be realized from the continuous presence of nitrogen in the soil environment over longer period of time. Indeed, research has shown as much as 11% loss in total nitrogen from NOx release from the soils. Therefore, the balance of nitrogen using the main bulk water path can be bounded within a ±10% error rate.

An important aspect of understanding the dynamics of nitrogen within the soil profile is that its temporal characterization is driven by events, namely irrigation events and the resultant soil moisture infiltration conditions that drive the mobilization and transport of nitrogen. Each of these events creates a dynamic environment in which rapid changes in nitrogen speciation and mobilization can occur. The physical transport of soluble nitrogen species is dominated by the liquid flow of moisture through the soil profile. Therefore, within each level of the soil profile, measurements over time are necessary in order to assess the changes in nitrogen levels and the likely outcome in terms of nitrogen inputs and losses. Determination of the species of nitrogen is can also be key to the assessment of nitrogen fate.

The monitoring system described in detail in the incorporated International Application No. PCT/US12/27588, produces relevant measures of nitrogen inputs for the bulk irrigation supply, fertigation supply when operated, and at various depths through the soil profile representing the nitrogen levels in the soil pore water environment during such events. The distinction in nitrogen species is determined by the sampling device through the use of various ion selective electrodes (ISEs).

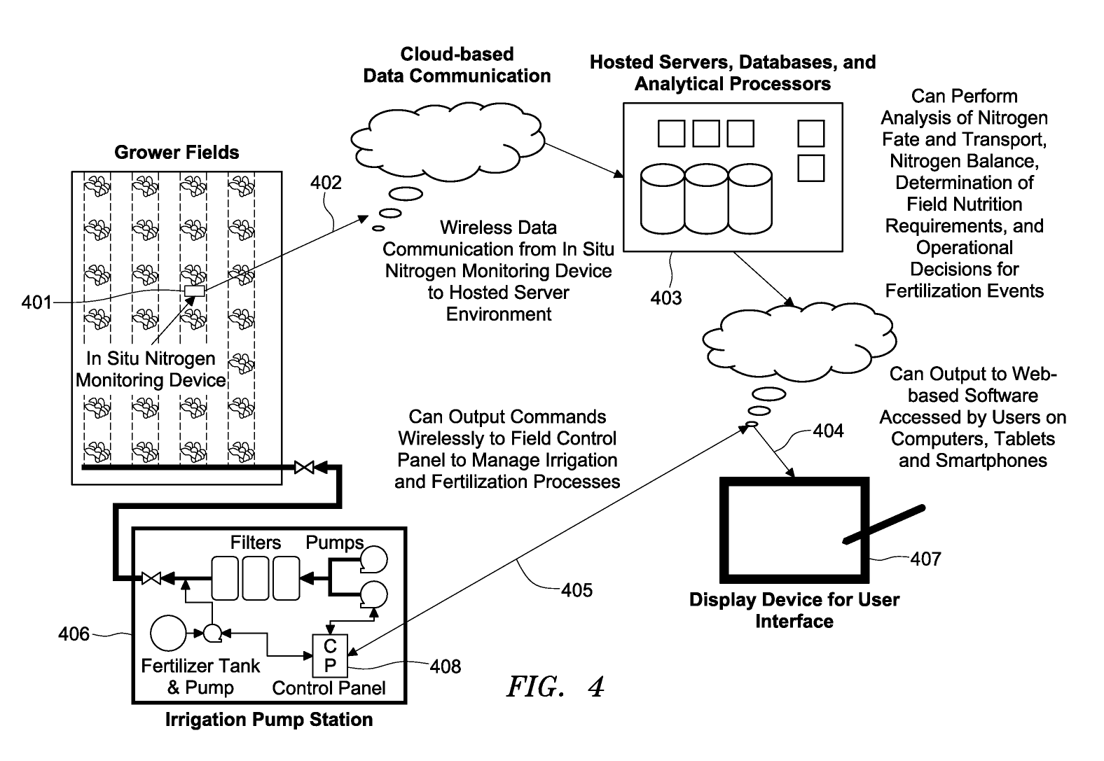

A description of the total system to drive data on nitrogen fate and transport from agricultural fields to control of nitrogen applications to the field is depicted in FIG. 4. Field measurement data captured by the sampling device 401 is delivered wirelessly 402 to centralized computer servers where the data is processed, converted into forms that are understandable to users, placed in data management systems, and analyzed for changes in nitrogen species 403. Using the outcome of an effective root zone determination (see below), the nitrogen content data produced by the field sampling system can be further analyzed to determine the nitrogen losses attributable to plant uptake, excessive infiltration, and volatilization. These results are provided to users 407 in such a way that users can control up-coming fertilization events through remote automation and control of their irrigation pump stations 408.

By determining over time the amount of nitrogen, throughout the depth of the soil profile, key parameters of nitrogen availability, consumption, and environmental fate can be determined. As shown in FIG. 3, nitrogen balances 336 are possible and embodiments disclosed herein enable the determination of one or more of the following:

Total Nitrogen (Nt). At any point in time and at any given level in the soil profile, the total nitrogen refers to that nitrogen likely to be available over time in the soil-water-air environment. This definition preferably ignores the “permanently fixed” nitrogen sorbed onto soil particles. Total Nitrogen content is determined as the sum of all of the nitrogen found in the bulk water, that adsorbed to the soil but able to be released in a near-time frame (e.g., days or weeks), and that remaining in the soil pore environment as a gas, and may be shown as:

Nₜ=Bulk water Nₜ+Soil absorbed Nₜ+Gaseous Nₜ (1)

Soil Adsorbed Nitrogen. The amount of soil that remains in a sorbed form on soil particles. This is determined by inference, and represents the differential in nitrogen sources applied versus loss from the environment over time, and may be shown as:

Soil Adsorbed Nₜ=Sum(ΔNₜ) Irrigation Supply−Sum(ΔNₜ) Bulk Pore Water−Sum(ΔNt) NOx Loss)/Δt (2)

Plant Nitrogen. The amount of nitrogen uptaken by plant root systems. The losses of available nitrogen—defined as the total of the nitrate and nitrite levels—over time are associated with plant uptake as these species are not sorbed onto soil particles are reduced nitrogen for volatilization is bounded by the expected range of nitrogen losses from this source. Plant nitrogen may be shown as:

Plant Nt=SUM(Δ (NO₃/NO₂)ₜ/Δt) in Active Root Zone of the Soil Profile (3)

Groundwater Nitrogen Risk. This is the risk that nitrogen contamination sources for underlying groundwater could originate from the nutrition management program operated by growers. The nitrogen risk is inferred by the amount of nitrate and nitrite that passes below the effective root zone of a crop, and therefore is highly likely to continue to migrate down to underlying aquifer systems. Groundwater nitrogen risk may be represented by:

Groundwater N Riskₜ=SUM(Δ (NO₃/NO₂)ₜ/Δt)below the Active Root Zone of the Soil Profile (4)

Climate Nitrogen Risk. This is the risk that nitrogen sources used for agriculture can contribute to atmospheric NOx and become a climate-change contamination source. Changes in nitrous oxide species in the general atmosphere and the soil pore gas environments tracked over time contribute to this risk, and can be represented by:

Climate N Riskₜ=SUM(Δ(NOx)ₜ/Δt) (5)

These analytics are provided from systems used for monitoring the environment of agricultural settings. Use of that data provides growers with clear and unambiguous information about the fate and transport of nitrogen in agricultural environments.

FIG. 4 depicts an example embodiment of a system 400 used for agricultural monitoring and/or management. System 400 integrates in situ (i.e., directly in the soil matrix) field monitoring for nitrogen compounds with devices that can be used to convey information about the nitrogen levels to farmers as well as devices that control the operation of irrigation pump and fertilization stations.

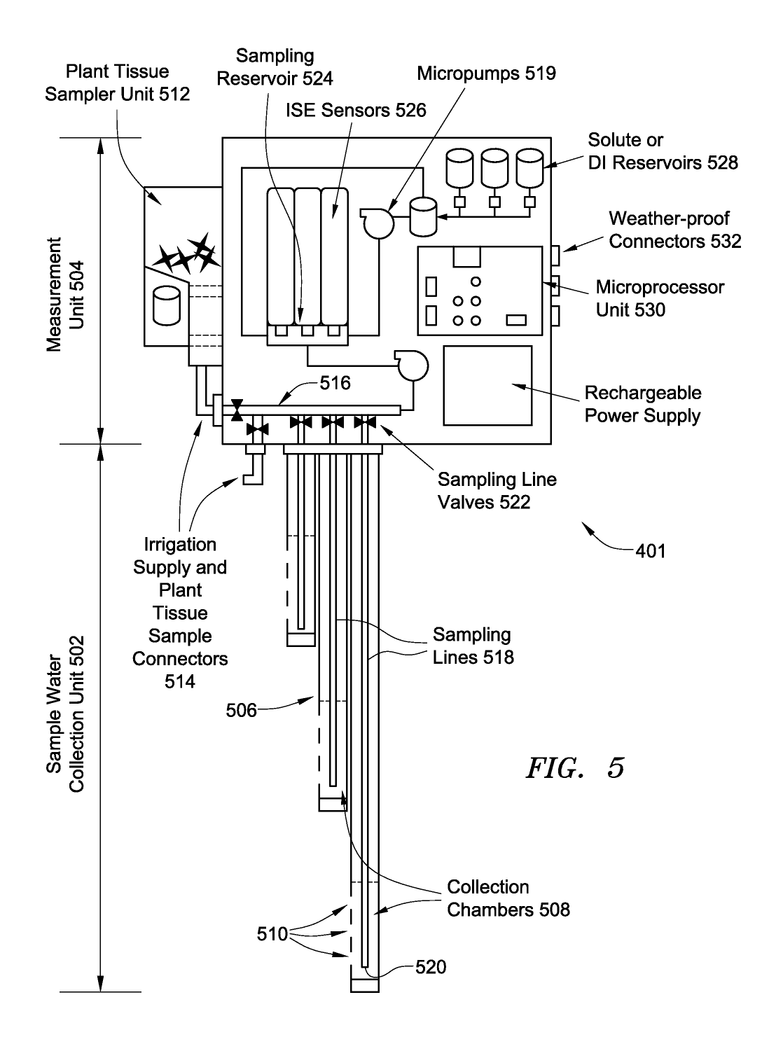

The in situ nitrogen monitoring device 401 is described separately in the incorporated International Application No. PCT/US12/27588 and an example embodiment of which is also described with respect to FIG. 5.

In general, the monitoring device (or field sampling device) 401 includes a sample collection unit 502 and a measurement unit 504. The sample collection unit 502 and the measurement unit 504 may be comprised of one physical integral unit, or located in close proximity to each other. Alternatively, the sample collection unit 502 and the measurement unit 504 may be located physically separate or remote from each other and coupled together via tubing, piping and the like.

The sample collection unit 502 for soil pore water sample collection is generally comprised of an elongate assembly or tube 506 with one or more collection chambers 508 formed therein and located at various depths along the assembly 506. (Soil pore water is broadly defined as water that is found to occupy the void spaces between and around soil particles.) In some embodiments the assembly 506 is preferably made of a rigid material for durability, however other materials may also be used. The unit 502 is installed in the soil, although any portion of the device can be installed directly in (or beneath) the soil, including the sensors and the entire measurement unit 504. Generally an augered hole of the same or similar size as the diameter of the tube 506 is created or bored into the soil environment in an agricultural field or other desired location. The tube 506 is then placed into a bored hole. The depth of the hole will vary depending on the type of agricultural use and by the crop type. In one example the depth is in the range of approximately 6 to 36 inches.

The walls of the collection chambers 508 include holes, openings or vents 510 along the length and/or circumference of the sample collection unit 502 at spaced intervals to allow soil pore water to flow into the collection chambers 508 located at the same depth. Alternatively the walls of the collection chambers 508 may be porous. Typically the openings are configured such that water may flow into the collection chambers 508, while soil, rock and other solid material does not pass through. Water seeps into the collection chambers 508 during wetting events. For purposes of this description a wetting event is defined as irrigation, rainfall, or both.

The sample collection unit 502 is coupled to the measurement unit 504. In one example, the sample collection unit 502 is connected or coupled to the measurement unit 504 by micro-tubing and miniaturized connectors. The sample collection unit 502 generally integrates the flow of one or more samples to ISE sensor(s) from either an external water source (such as the irrigation supply or rainfall), the soil pore water, or with an attachment to the unit, samples derived from a plant tissue processing unit 512. Additionally, samples may be obtained from the irrigation supply via an irrigation supply port 514. To manage the flow of samples, a sampling assembly is provided comprised broadly of a manifold 516 and a plurality of sampling lines 518. One or more micro-pumps 519 are coupled to the manifold and sampling lines. Generally, each of the sampling lines 518 is independently coupled to the manifold 516 and has an open distal end 520. This open distal end 520 of at least one of said sampling lines 518 extends into each of the collection chambers 508 to draw soil pore water samples from each of the collection chambers up through the manifold 516 via valves 522 and into a sampling reservoir 524. Once samples have been drawn up into the sampling reservoir 524, detection of one or more nutrients in the samples using one or more ISE sensors housed in an ISE sensor chamber 526 may begin. Each ISE sensor is preferably capable of sensing differences between nutrient species, e.g., capable of distinguishing between nitrogen species.

In some embodiments, the measurement unit 504 preferably houses the ISE sensor chamber 526, micro-pumps 519, all electronics, battery power supply, and reservoirs of various fluids as needed for analyses, including DI water (described in more detail below). However, other configurations are possible within the scope and spirit of the present teachings.

When the ISE sensors are not in a measurement state, a continuous supply of DI water from DI reservoir 528 is re-circulated through the ISE sensor chamber 526 to maintain a wetted environment for the ISE sensors, when needed.

One or more sensors (not shown) adapted to measure moisture levels in the soil are preferably associated with (e.g., included within or coupled with) the field sampling device 500. These sensors are preferably positioned at different depths within the soil and are capable of detecting the moisture level in the soil at that depth. The sensors can be included within a housing of the field sampling device 500, e.g., such as the main physical housing of the measurement unit 504, or they can be located outside of the monitoring device housing and coupled with the field sampling device 500 by way of, e.g., an electrical cable.

The field sampling device 500 includes a microprocessor unit 530 configured to carry out sample initiation, perform ISE sample measurement, perform soil moisture measurement, processing of the ISE and/or soil moisture measurements, perform data acquisition and transmission, manage the power supply delivery, control operation of the micropumps, make sensor data recordings and data transmission to data acquisition systems using standard communication protocols, and end the monitoring session, among other functions. Valve controls are managed by the measurement unit microprocessor in terms of sample collection frequency and clean sample flushing with DI water between samples, as needed.

The microprocessor 530 may include weather-proofed connectors 532 coupled thereto to enable additional functions such as: solar panel recharge of the power supply; connection to the data acquisition and transmission unit, and receiving in-coming signals from additional sensors that may be useful for the operational logic of the measurement unit 504.

Referring back to FIG. 4, the in situ nitrogen monitoring device 401 is adapted to collect data (e.g., representative of the level of moisture in the soil in which the monitoring device is implanted and/or representative of the content or amount of one or more species of nutrients). This data can be packetized and delivered wirelessly to a data management system 403 over a communications path 402. The communications path is preferably a wireless path (or link, or channel) emanating from the monitoring device 401, but can also include wireline portions before the data reaches the data management system 403. Alternatively, the communications path 402 can be entirely wireless or wireline between device 401 and system 403.

Data management system 403 is hosted on computer servers. The servers are comprised of a number of processor units which can support databases, a multitude of data processing engines, and a variety of other services including the hosting of browser-based software that users can access using local devices. The data management system can also include other analytical processors outside of those resident in the servers.

Data output by the data management system can then be delivered over another communications path 404 to a user device 407 having a graphical user interface permitting use of a web browser-based system. Examples of the user device 407 can include a personal computers, laptop, tablets, or smartphones. An example of the graphical user interface is a touch screen or a typical mouse/keyboard/display combination. The data to the user device can be a data message containing information about the nutrient level in the soil for the user. Alternatively, the resulting data can be sent over communications path 405 to a field control device where commands for the operation of irrigation and fertilizer pumps can be implemented. One example of this is a control panel 408 at an irrigation pump station 406. The transmission can be in the form of a command to the irrigation pump station to perform (or schedule the performance of an irrigation or fertigation event to increase the water or nutrient level of the soil.

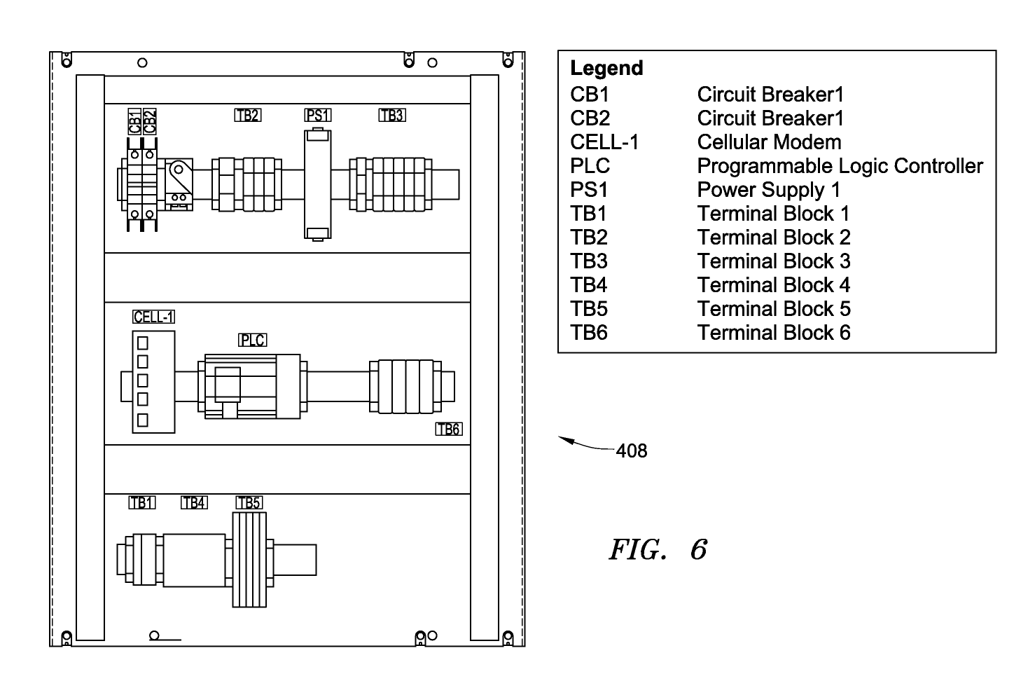

Process control panels are commonly used in industry, including agriculture, for scheduled or automated control of equipment. An example embodiment of a control panel is shown in FIG. 6. The processing of nitrogen data will enable commands for the automated operation of irrigation pumps and fertilizer pumps to be delivered to local control panels. In this embodiment, the control panel 408 includes one or more terminal block connectors (TB1-TB6) for electronically delivering commands to local equipment and collecting data from sensors deployed at the pump stations. Also included are one or more circuit breakers (CB1-CB2) to protect the electrical equipment in the panel, one or more power supply converters to enable the system to perform with a range of power supply requirements, one or more programmable logic controllers (PLC) equipped with digital and/or analog input and output terminals that can be wired to communicate with the equipment in the field, and a cellular (or wireless) telemetry unit (CELL-1) that interacts with the PLC in delivering and receiving wireless commands and data.

One of the key issues in partitioning the fate and transport of nitrogen is the determination of the effective root zone (ERZ) in the soil profile. Determination of the ERZ is important to the determination of nitrogen-specie movement to plants and the risk of contaminating underlying groundwater resources. An algorithmic approach is used with the specialized system described herein to determine the ERZ of the soil profile based on real-time data captured from the field for soil moisture changes over time. Those depths in the soil profile below the ERZ comprise the “deep water bank” (DWB) where resident soil moisture stored in this section of the profile is available as crops mature and the roots extend to deeper depths. Changes in nitrate and nitrite levels over time within the ERZ profile represent nitrogen losses due to plant uptake.

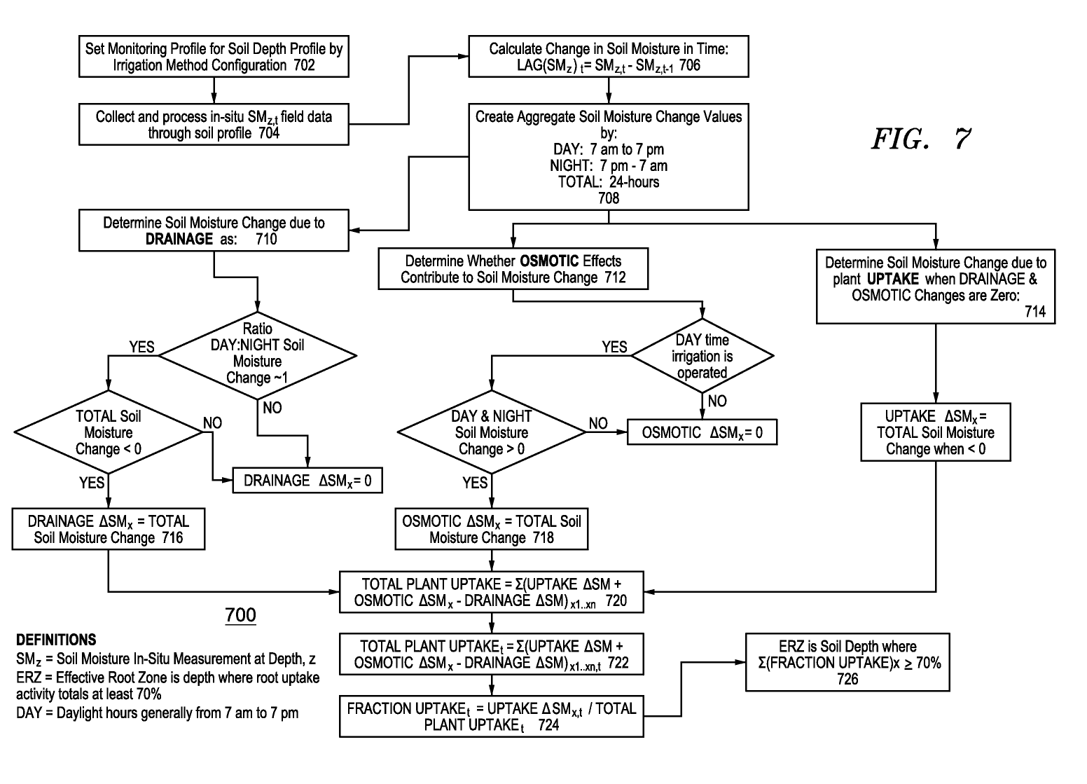

Example embodiments of methods for determining the ERZ for a soil profile are described below. These methods are understood to be implemented primarily on the data management system, which can be adapted to perform the various steps of each of the example methods. An example embodiment of a method 700 for determining the ERZ depth is illustrated in FIG. 7. Defining the soil depth profile monitored at step 702 and having collected soil moisture data throughout that depth profile at step 704, the following steps are performed to determine the ERZ:

Calculate the differential in soil moisture within each time step measured to determine the change in soil moisture at step 706. When this change is negative, soil moisture is decreasing in time; and when positive, the soil moisture is increasing in time.

Segment the change in soil moisture levels found by whether it occurs during daylight hours (assume generally 7 am-7 pm) when plants are productive and nighttime hours (assume generally 7 pm to 7 am) when plants are generally less active or inactive at step 708.

Once the change in soil moisture values are segmented by daytime and nighttime hours, the mechanisms by which the change has occurred is determined. Of particular advantage embodiments of the present invention provide for determining whether the change in soil moisture values is predominately due to drainage, osmotic effect or plant uptake.

Specifically, at step 710, determine whether the dominant form of water loss for the day is drainage within a given soil profile. When drainage dominates, the change in soil moisture across daylight and nighttime hours are very similar. Therefore, the ratio of the sum of daylight changes with the sum of nighttime changes should be near unity.

Determine if osmotic behavior in plant uptake of soil moisture is contributing to the total soil moisture uptake levels at step 712. When roots have to pull water to them to get access, soil moisture changes at a given depth can be positive in daylight hours and still represent a water loss, and the drainage over night represents a net loss from the soil profile. Note that on days when irrigation is practiced, the ability to determine osmotic behavior is eliminated, so limits on the analysis are placed to compensate for irrigation activity.

Determine the Plant Uptake of soil moisture at various depths within the profile based on the soil moisture changes in daylight hours at step 714 when drainage is not flagged (step 716) nor irrigation is occurring. If osmotic behavior is determined to occur at a given depth in the profile, the soil moisture change due to osmotic behavior is the plant uptake of soil moisture 718.

The total plant uptake is determined for the entire soil profile by summing all of the plant uptake levels found for each depth interval at steps 720, 722. This can be expressed for any point in time or for all aggregated periods of time.

Determine the fraction of plant uptake at each profile depth level by calculating the ratio of plant uptake at a given depth and the total plant uptake for the entire soil profile at step 724.

The ERZ is determined at step 726 by accumulating the fraction of root zone activity found in step 724 until, e.g., at least 70% of the total is achieved. At that depth, the ERZ occurs.

Another example embodiment of a method for determining ERZ recognizes that the determination of the ERZ is inextricably tied to the agricultural monitoring system itself. This embodiment recognizes that the receipt of data at the data management system from the monitoring device over the communications path is an inseparable aspect of the method. The method is for determining an effective root zone (ERZ) for a crop in an agricultural monitoring system, where the agricultural monitoring system includes (a) a data management system hosted on a server and (b) a monitoring device having at least one sensor and at least partially located within soil.

The monitoring device is adapted to measure a moisture level of the soil at a plurality of different depths (two or more, but preferably four to five), and is adapted to wirelessly transmit data representative of the moisture level of the soil at the plurality of different depths. Preferably, the monitoring device is adapted to collect data from a wide range of depths, with at least one of those depths being deeper than where the ERZ is expected to lie. Because the root zone changes with the growth process, the monitoring device is preferably adapted to collect data at a range of depths that will capture the movement of the ERZ, and enable monitoring of the ERZ movement during the crop season. The system can be capable of determining the ERZ to a precision within about ±10% of the ERZ depth range. For permanent or deep rooted annual crops, e.g., having an ERZ of between 24 and 36 inches, a suitable precision could be about ±3 inches. For row crops with more shallow root zones, e.g., having an ERZ of between 12 and 24 inches, a suitable precision could be about ±1.5 inches.

The example method is performed primarily at the data management system. First, a communications path between the data management system and the monitoring device is established. The communications path is preferably at least partially wireless. Next, data representative of the moisture level of the soil at each of the plurality of different depths is received at the data management system from the monitoring device.

This data can be collected and/or communicated in “real-time.” For instance, in a typical agricultural context, soil moisture varies on an hourly basis. Appropriate collection intervals in that context are on the order of minutes. A fifteen minute interval may provide enough granularity to recognize variations in the soil moisture level, and a five minute interval provides three times that. Other variables may have a rate of change measured on the order of days, in which case hourly intervals between analyses of different collections can be sufficient. Preferably, the interval between a first analysis and the next analysis is smaller than the rate of change of the variable being analyzed by enough of a margin so that, as the analyses continue over time, non-negligible changes in the variable (e.g., nutrient content, etc.) can be identified. Those of ordinary skill in the art will readily recognize those changes that are non-negligible for a particular variable in a particular setting. Collection or analysis of samples in this fashion, or communication of data related to those samples in this fashion, is referred to herein as occurring in “real-time.”

The data management system preferably includes one or more analytical processors and one or more databases, and is also preferably hosted on servers, that will typically be remote from the monitoring device. Once the data management system has received the data from the monitoring device (either through one transmission or multiple transmissions over the course of time), the data management system can broadly determine a root uptake value indicative of the extent to which root uptake occurs at each of the plurality of different depths and then determine an ERZ value for the crop based on the determined root uptake values and a predetermined ERZ criteria.

More particularly, the data management system can determine, for each of the plurality of different depths, whether each of a plurality of incremental changes in soil moisture is relevant for root uptake. These incremental changes can be changes that are measured throughout the course of a day. For instance, they can be changes recorded every ten minutes, every hour, every two hours, and so forth. A change may be considered relevant for root uptake if, for example, that change is a depletion in soil moisture occurring during daytime hours. If the change is relevant for root uptake it can be flagged to identify it as such. Changes that are not relevant for root uptake can also (or alternatively) be flagged with a different flag.

Next, for each of the plurality of incremental changes in soil moisture determined to be relevant for root uptake, the data management system determines whether that change in soil moisture is due to drainage. This can be done by comparing that change with an expected drainage rate of change given the soil properties. If due to drainage, then the change can be flagged as a drainage event rather than a root uptake event.

Then, the data management system can determine, for each of the plurality of different depths, whether root uptake occurs based at least on those incremental changes in soil moisture occurring at that particular depth that are not due to drainage. This may include osmotic soil moisture changes. This can be accomplished, for example, by evaluating whether those incremental changes in soil moisture occurring that are not due to drainage at a particular depth represent accumulation during daytime and depletion during nighttime. For instance, if the incremental changes during the day represent accumulation (e.g., positive) and the incremental changes during the night represent depletion (e.g., negative) then the net daily change in soil moisture represents the root uptake loss for that depth.

The data management system can then determine a root uptake value for each of the plurality of different depths where root uptake occurs, for instance, by aggregating the total moisture change due to root uptake over a specified time period (e.g., twelve hours, a day, two days, a week, etc.) for each depth. Then, an ERZ value for the soil, based on the determined root uptake values and a predetermined ERZ criteria, can be determined by, for instance, determining the total daily root uptake and the portion of that total contributed by each depth and checking those values against the ERZ criteria. The ERZ value can be the range of depths at which the effective root zone is present.

The predetermined ERZ criteria can be a fraction of the total root uptake. The fraction can be exact, rounded, or approximate. The selection of the fraction at which ERZ occurs is variable based on the user's preference. An preferred example is 70%, or approximately 70%. However, other values can be used, such as greater than or equal to about 50%, between about 60% and about 80%, between about 65% and about 75%, and between about 69% and 71%.

In this and all embodiments, the ERZ value can then be used as a basis for determining a nutrient level for the soil. In the case where the nutrient level is a nitrogen level, then the nitrogen level can be a level of any one or more of the following (or a level of the nitrogen contained in any one or more of the following): nitrate, nitrite, ammonium, inorganic nitrogen, organic nitrogen, gaseous nitrogen or nitrogen bound to soil. This nutrient level determination can then be used by the farmer or other user to make decisions as to whether to irrigate, fertigate, or fertilize the soil to add water or the nutrient for which the determination was made to the soil. (Or alternatively, to arrest the addition of water or the nutrient.) For instance, the data management system can output (a) a command to a control panel of an irrigation pump station for modifying the nutrient level or (b) a data message about the nutrient level to a user device having a graphical user interface. If so, a communications path with the irrigation pump station or user device is preferably first established.

It is important to note that the example method just described can also include actions performed by the monitoring device or the irrigation pump station.

With the determination of the ERZ, the nitrogen uptake by plants can be discerned from the nitrogen losses that are likely to drive contamination potential for underlying ground water. By determining the ERZ, the system can also evaluate the likely micro-ecology suitable for nitrogen conversion to NOx species, as these conversions require microbiological interactions with nitrogen compounds.

Various embodiments of the present invention include one or more, or all of the following components: an in-depth and rich database of real-time and periodic monitoring for soil moisture and nitrogen compounds from agricultural fields and their irrigation water resources; methods by which the data are segmented by soil depth profile, monitored medium, and time periods of representative data; methods to determine the total applied water and nitrogen in agricultural settings over time; methods to determine changes in soil moisture that can be attributed to soil pore water drainage and plant uptake; and methods to determine nitrogen uptake by plants versus contributing to environmental contamination risks.

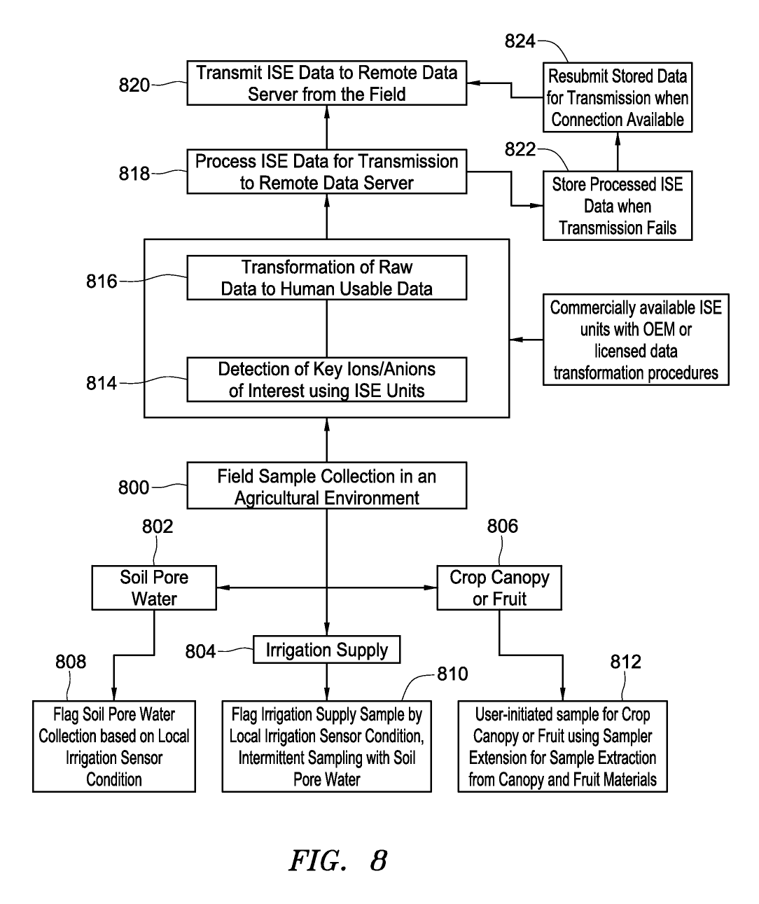

One exemplary method of operation of the field device is illustrated in the flowchart shown in FIG. 8. In general, collection of field samples from an agricultural environment is provided at step 800. The field samples may be obtained from a variety of sources, specifically any one or more of: soil pore water 802, the irrigation supply 804, and the crop canopy or fruit 806. In the exemplary embodiment, soil pore water collection is initiated based on defined event trigger conditions—either automated based on local irrigation sensors or more manual/interval based trigger conditions, step 808. Irrigation supply sampling can be initiated either by (1) sensors that monitor the irrigation system operation that are linked (in communication with the field sampler device) or directly connected to the device or, (2) by user-initiated events (manual operation) at step 810. Crop canopy or fruit samples are initiated by the user as shown in step 812.

Once samples are collected, detection of ions or anions of interest are detected using ISE sensors as shown in step 814. The raw data from the ISE sensors is then transformed to human usable data at step 816.

Next, the transformed data is processed for transmission to one or more remote data servers at step 818. Finally the data is transmitted to the one or more remote data servers at step 820. Data is stored in the event of transmission failure and then resubmitted once connections are available, as shown in steps 822 and 824, respectively.

These embodiments can be used by growers to improve their nutrient management needs, reduce cost of crop production, and improve productivity of crops. Growers and policy makers alike can use the methods and systems of embodiments of the invention to determine appropriate best practices for managing off-farm migration of nutrients to minimize their potential for creating environmental damage. Based on the embodiments disclosed herein, the user can discern or estimate by deduction one or more and preferably all of the following forms of nitrogen in an agricultural environment: (a) Nitrate/Nitrite in soil pore water; (b) ammonium in soil pore water; (c) nitrogen—inorganic and organic—in plant tissues and fruits; (d) gaseous nitrogen release from the soil environment; and (e) nitrogen bound to the soils. Preferably, direct measurement of the first four forms of nitrogen in agricultural environments is made.

It should be noted that, while various embodiments have been described, those embodiments should not be viewed as being unrelated. In fact, each of the embodiments described are intended to complement each other, and any feature, element, step, or aspect of an embodiment can be combined with any other embodiment unless explicitly stated otherwise. For instance, a step recited in one method embodiment can also be performed in a separate method embodiment, and a structural component described in one apparatus embodiment can be included within another apparatus embodiment. Similarly, although numerous features, elements, steps, or aspects of each embodiment are described, they are not essential to that embodiment unless explicitly stated. In other words, it is intended that any feature, element, step, or aspect of an embodiment can be claimed by itself with the omission of any or all other features, elements, steps, or aspects of that embodiment, unless explicitly stated otherwise.

It should also be noted that various embodiments are described herein with reference to one or more numerical values. These numerical value(s) are intended as examples only and in no way should be construed as limiting the subject matter recited in any claim, absent express recitation of a numerical value in that claim.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

1. A method of determining an effective root zone for a crop in an agricultural monitoring system, wherein the agricultural monitoring system comprises (a) a data management system hosted on a server and (b) a monitoring device associated with at least one sensor and at least partially located within soil, the monitoring device being adapted to measure a moisture level of the soil at a plurality of different depths, the monitoring device also being adapted to wirelessly transmit data representative of the moisture level of the soil at the plurality of different depths, the method comprising the following steps performed by the data management system:
receiving data from the monitoring device, wherein the data is representative of the moisture level of the soil at each of the plurality of different depths;
determining, for each of the plurality of different depths and based on the received data, whether each of a plurality of incremental changes in soil moisture is relevant for root uptake;
determining whether each of the plurality of incremental changes in soil moisture determined to be relevant for root uptake are due to drainage;
determining, for each of the plurality of different depths, whether root uptake occurs based at least on those incremental changes in soil moisture occurring at that particular depth that are not due to drainage;
determining an amount of root uptake for each of the plurality of different depths where root uptake occurs; and
using at least the determined amounts of root uptake and a predetermined effective root zone criteria, determining an effective root zone for the crop.

2. The method of claim 1, further comprising determining a nitrogen level for the soil based on the effective root zone for the crop.

3. The method of claim 2, wherein the nitrogen level is a level of any one or more of: nitrate, nitrite, ammonium, inorganic nitrogen, organic nitrogen, gaseous nitrogen or nitrogen bound to soil.

4. The method of claim 2, further comprising outputting (a) a command for modifying the nitrogen level to a control panel of an irrigation pump station or (b) a data message about the nitrogen level to a user device having a graphical user interface.

5. The method of claim 4, further comprising establishing a communications path with the irrigation pump station prior to outputting the command to the control panel of the irrigation pump station, the communications path being at least partially a wireless communications path terminating at the control panel.

6. The method of claim 4, further comprising establishing a communications path with the user device prior to outputting the data message to the user device, the communications path being at least partially a wireless communications path terminating at the user device.

7. The method of claim 4, wherein the user device having the graphical user interface is a laptop computer, a tablet computer, or a smartphone.

8. The method of claim 4, wherein the graphical user interface comprises a touchscreen.

9. The method of claim 4, wherein the command to the control panel of the irrigation pump station is a command to schedule an irrigation event or a fertigation event.

10. The method of claim 4, wherein the command to the control panel is a command to perform fertigation based on a nitrogen level in the soil profile determined using the effective root zone for the crop.

11. The method of claim 1, wherein each of the plurality of incremental changes in soil moisture are relevant for root uptake if each change is a depletion in soil moisture occurring during daytime.

12. The method of claim 1, further comprising flagging each of the plurality of incremental changes in soil moisture that is determined to be relevant for root uptake.

13. The method of claim 1, further comprising determining, for each of the plurality of incremental changes in soil moisture determined to be relevant for root uptake, whether that change in soil moisture is due to drainage by comparing that change with an expected drainage rate.

14. The method of claim 1, further comprising determining, for each of the plurality of different depths, whether root uptake occurs by evaluating whether those incremental changes in soil moisture occurring that are not due to drainage at a particular depth represent accumulation during daytime and depletion during nighttime.

15. The method of claim 1, further comprising determining a root uptake value for each of the plurality of different depths where root uptake occurs by calculating a net daily change in soil moisture for each of the plurality of different depths where root uptake occurs.

16. The method of claim 1, wherein determining an effective root zone for the crop is performed by accumulating the determined root uptake values into a total daily root uptake and determining at which depth a predetermined fraction of the total daily root uptake occurs.

17. The method of claim 16, wherein the predetermined effective root zone criteria is approximately seventy percent of the total daily root uptake.

18. The method of claim 1, wherein the plurality of depths is four or five depths.

19. The method of claim 1, further comprising establishing a communications path with the monitoring device prior to receiving said data from the monitoring device, the communications path being at least partially a wireless communications path terminating at the monitoring device.

20. The method of claim 1, wherein the data management system hosted on the server comprises a database and an analytical processor.

21-62. (canceled)
